(19) United States
(12) Patent Application Publication
(10) Pub. No.: US 2020/0192889 A1
(43) Pub. Date: Jun. 18, 2020

(54) A method of building gut microbiota database and a detection system of gut microbiota

(71) Applicant: GenoRx Co.,Ltd, Taipei City (TW)

(72) Inventor: CHIH-MIN CHANG, Taipei City (TW)

(21) Appl. No.: 16/638,912

(22) PCT Filed: Aug. 16, 2017
(86) PCT No.: PCT/CN2017/000532

§ 371 Date: Feb. 13, 2020

(51) Int. Cl. G06F 16/23; G06F 16/28; G06F 16/22; G16H 10/40; G16H 70/60; G16H 50/30; G16H 50/20; G16B 30/10; C12Q 1/689; C12N 15/10

(52) U.S. Cl. CPC G06F 16/2379; G06F 16/285; G06F 16/2228; G16H 10/40; C12N 15/1093; G16H 50/30; G16H 50/20; G16B 30/10; C12Q 1/689; G16H 70/60

(57) ABSTRACT

A method of building gut microbiota database and a detection system of gut microbiota are disclosed. Specifically, the method is to provide an index for evaluating host health in vitro. Moreover, the detection system of gut microbiota comprises computer system that can analyze and classify the gut microbiota to generate useful information for building the gut microbiota database.

### TECHNICAL FIELD OF THE INVENTION

The present invention relates to a method of building gut microbiota database and detection system of gut microbiota. In particular, the method of building gut microbiota database is to provide the gut microbiota database with quantifiable indexes for evaluating host heath in vitro.

### BACKGROUND OF THE INVENTION

Recently, medical research found that human health and disease closely relate to gut microbiota. However, there is still no systematic way to analyze or research the relationship between host health and the gut microbiota exist in the host in vitro and further evaluate the host health.

Based on the aforementioned description, there is a emerge request to develop a method or system regarding systematic evaluating gut microbiota in vitro for applying in preventative and precision medicine.

### SUMMARY OF THE INVENTION

In one objective, the present invention provides a method of building gut microbiota database. The method comprises following steps.

Step 1: Input gene sequencing information of gut microbiota to a computer system comprising a metagenomics analyzing software, a known gut microbiota gene database, and a disease related gut microbiota database.

Step 2: Classify species of the gut microbiota with the computer system. The step 2 includes following steps.

Step 2-1: Execute the metagenomics analyzing software to generate group information of the gut microbiota by alignment and blasting algorithm, wherein the alignment and blasting algorithm calculate sequence similarity of each gene sequencing information and group gut microbiota data into operational taxonomic units when the sequence similarity of each gene sequencing information of the gut microbiota is more than 97%.

Step 2-2: Correspond the operational taxonomic units of the gut microbiota to taxonomic information stored in the known gut microbiota gene database.

Step 2-3: Output the species of the gut microbiota and abundance of the species of the gut microbiota.

Step 3: Cross-compare the species of the gut microbiota with the disease related gut microbiota database, and output classifying information of related gut microbiota by the computer system. The disease related gut microbiota comprises cancer related gut microbiota, cardiovascular disorders related gut microbiota, metabolism related gut microbiota, autoimmune related gut microbiota, gastrointestinal disorders related gut microbiota or mental/psychiatric related gut microbiota; and further classifying the related gut microbiota into positively correlated or negatively correlated gut microbiota.

Step 4: Output test value R(x) of the species of the gut microbiota that correlated to each diseases by the computer system.

The test value R(x) is obtained from following equation

$$R ( x ) = \frac{10}{- {log}_{10} x}$$

x is the abundance of the species of the gut microbiota.

Step 5: Output reference value range of the species of the gut microbiota that correlated to each diseases by the computer system, wherein the reference value range of the species of the gut microbiota is calculated by following equation.

$$Reference value range of the species of the gut microbiota = \frac{10}{- {log}_{10} ( x - 2 std )} ∼ \frac{10}{- {log}_{10} ( x + 2 std )}$$

x is the abundance of the species of the gut microbiota.

std is standard deviation of the abundance.

Step 6: Output a seties of radar charts by the computer system, wherein the seties of radar charts include following information: the classifying information of related gut microbiota, the test value R(x) of the species of the gut microbiota and the reference value range of the species of the gut microbiota that correlated to each disease by the computer system.

Step 7: Output a total protecting value (V) of the correlated gut microbiota by the computer system, wherein the total protecting value (V) of the related gut microbiota is an index for evaluating host health and calculated by following equation.

(V)=P[1−(Aₚ/Aₚᵣ)]+N[(Aₙ/Aₙᵣ)−1]

P is weights of the positively correlated gut microbiota in the related gut microbiota, 0≤P≤1; N is weights of the positively correlated gut microbiota in all the related gut microbiota, 0≤N≤1; Aₚ is area of the polygon constructed by the test value of the positively correlated gut microbiota; Aₚᵣ is area of the polygon constructed by the reference range of the positively correlated gut microbiota; Aₙ is area of the polygon constructed by the test value of the negatively correlated gut microbiota; Aₙᵣ is area of the polygon constructed by the reference range of the negatively correlated gut microbiota.

Step 8: Build a gut microbiota database according outputting results from step 3 to step 7 by the computer system. The gut microbiota database contains the classifying information of related gut microbiota, the test value R(x) of the species of gut microbiota in the related gut microbiota, the reference value range of the species of gut microbiota in the related gut microbiota, the plot described in step 5, the total protecting value (V) of the related gut microbiota, evaluating index of diversity of the gut microbiota and enterotypes of the gut microbiota.

In one embodiment, the method further comprises a step of extracting bacterial DNA containing gut microbiota from a stool sample and a sequencing process with bacterial gene amplification methods.

In an representative example, the sequencing process with the bacterial gene amplification method comprises following steps: (1) Perform PCR on the bacterial DNA containing gut microbiota from the stool sample to amplify the bacterial DNA; (2) For the PCR above, use a pair of primers consisting of a forward primer and a reverse primer that complement to V3-V4 regions of 16 S rRNA gene among the bacterial DNA sequence, and amplify the V3-V4 sequence of 16 S rRNA gene of the gut microbiota to a length of about 550 bps; (3) Purify the V3-V4 sequence of 16 S rRNA gene of the gut microbiota and repeating step (2) and (3) to obtain a library with a length of about 630 bp; (4) Measure the size and concentration of the library by the analyzer and fluorometer, adjusting the concentration of the library, and adding the library onto a sequencing chip with complementary adaptors on surface of the sequencing chip; (5) Perform a bridge amplification by gene sequencing platform to amplify fluorescent detecting signals; and (6) Remove fluorescent markers and detect the fluorescent signals repeatedly to obtain gene sequencing information of the gut microbiota, wherein the gene sequencing information of the gut microbiota has a pair-end sequencing length of about 2*300 bp.

In one embodiment, if the total protecting value (V) of the related gut microbiota is more than or equal to zero, the computer system will output a result indicating normal host heath; and if wherein the total protecting value (V) of the related gut microbiota is less than zero, the computer system will output a result indicating abnormal host heath.

In one embodiment, the evaluating index of diversity of the gut microbiota is Shannon's diversity index which ranges from 1.86 to 4.89, if Shannon's diversity index of stool sample is more than or equal to 3.375, the stool sample is determined to a stool sample with high diversity of the gut microbiota; if Shannon's diversity index of stool sample is between 3.375 and 2.6175, the stool sample is determined to a stool sample with intermediate diversity of the gut microbiota; and if Shannon's diversity index of stool sample is less than or equal to 2.6175, the stool sample is determined to a stool sample with low diversity of the gut microbiota.

In one embodiment, the enterotypes of the gut microbiota comprises Prevotella, Bacteroides, Escherichia or Ruminococcus; and select the species of the gut microbiota having maximum test value of the species of gut microbiota to be the enterotypes of the gut microbiota in the stool sample.

In one embodiment, the gut microbiota database is save in a cloud storage and a gut microbiota detecting system.

In another objective, the invention provides a detecting system of gut microbiota. The detecting system of gut microbiota comprises, a sampler that collect stool sample, a gene information processing system, and a computer system. The gene information processing system comprises a gene library measuring unit and a gene sequencing unit. Typically, the computer system receive gene sequencing information output from the gene information processing system and execute the method described from paragraph [symbol] 0005 [symbol] to [symbol] 0030 [symbol] for building a gut microbiota database. The gut microbiota database comprises classifying information of related gut microbiota, test value R(x) of species of gut microbiota, reference value range of species of gut microbiota, analytical plots, total protecting value (V) of the related gut microbiota, evaluating index of diversity of the gut microbiota and enterotypes of the gut microbiota.

In one embodiment, the gene sequencing information is obtained by performing following steps: (1) extract bacterial DNA containing gut microbiota from a stool sample; (2) Perform PCR on the bacterial DNA containing gut microbiota from the stool sample to amplify the bacterial DNA; (3) For the PCR above, use a pair of primers consisting of a forward primer and a reverse primer that complement to V3-V4 regions of 16 S rRNA gene among the bacterial DNA sequence, and amplifying the V3-V4 sequence of 16 S rRNA gene of the gut microbiota to a length of about 550 bps; (4) Purify the V3-V4 sequence of 16 S rRNA gene of the gut microbiota and repeating step (2) and (3) to obtain a library with a length of about 630 bp; (5) Measure the size and concentration of the library by the analyzer and fluorometer, adjusting the concentration of the library, and adding the library onto a sequencing chip with complementary adaptors on surface of the sequencing chip; (6) Perform a bridge amplification by gene sequencing platform to amplify fluorescent detecting signals; and (7) Remove fluorescent markers and detect the fluorescent signals repeatedly to obtain gene sequencing information of the gut microbiota, wherein the gene sequencing information of the gut microbiota has a pair-end sequencing length of about 2*300 bp.

The detecting system of gut microbiota is applied to evaluate physiological conditions of a subject in vitro. The physiological conditions comprises physiological conditions of digestive system, physiological conditions of metabolic system, physiological conditions of immune system, physiological conditions of digestive tract cells, physiological conditions of central nervous system and/or physiological conditions of cardiovascular system.

Accordingly, the present invention provide the gut microbiota database with quantifiable indexes for evaluating host heath in vitro, and is able to apply in preventative and precision medicine.

### BRIEF DESCRIPTION OF THE DRAWINGS

FIG. 1 is a process flow diagram for building the invented gut microbiota database;

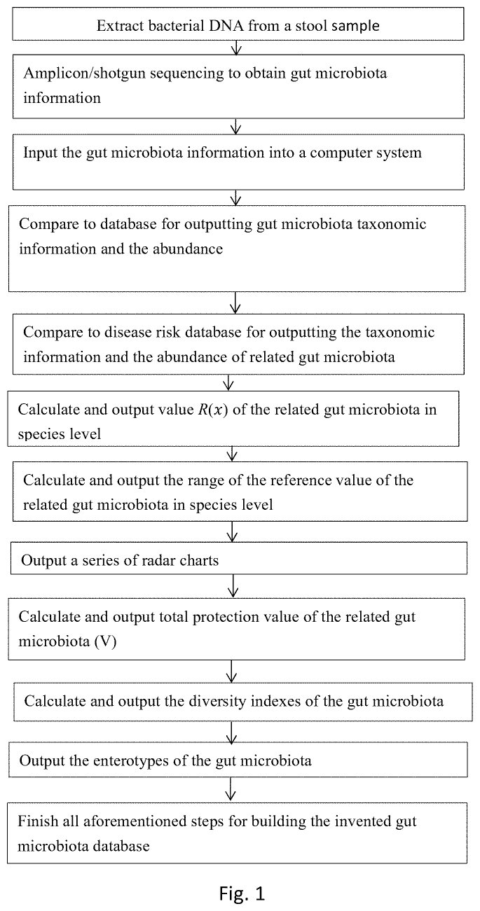

FIG. 2 is a radar chart of colorectal cancer related gut microbiota in a stool sample described in example 1;

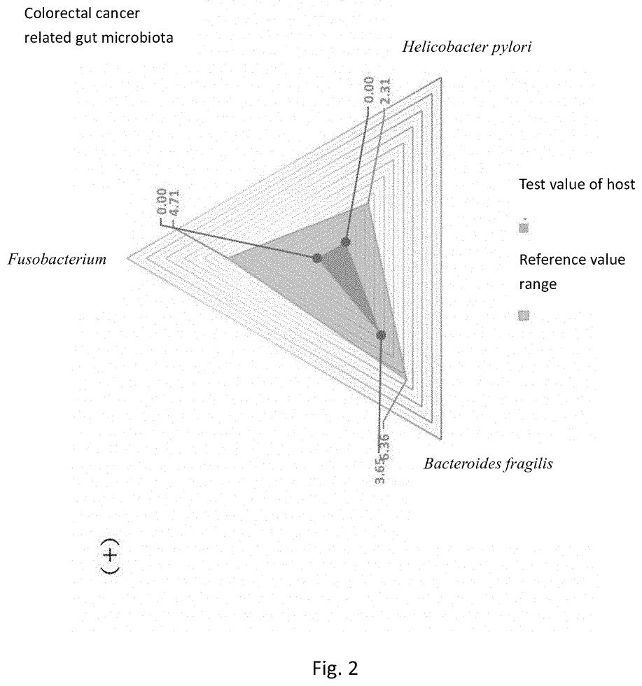

FIG. 3 is a positively correlated gut microbiota radar chart of atherosclerosis related gut microbiota in a stool sample described in example 1;

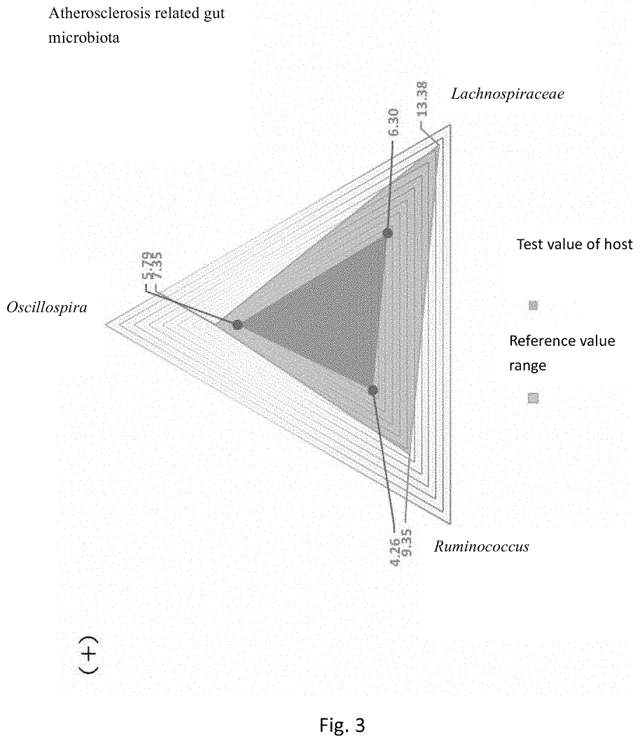

FIG. 4 is a negatively correlated gut microbiota radar chart of atherosclerosis related gut microbiota in a stool sample described in example 1;

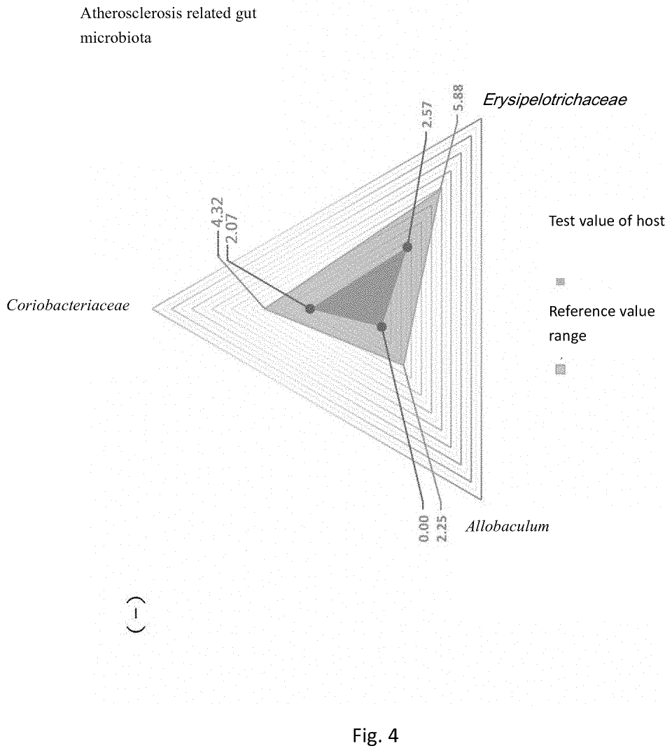

FIG. 5 is a positively correlated gut microbiota radar chart of type II diabetes related gut microbiota in a stool sample described in example 1;

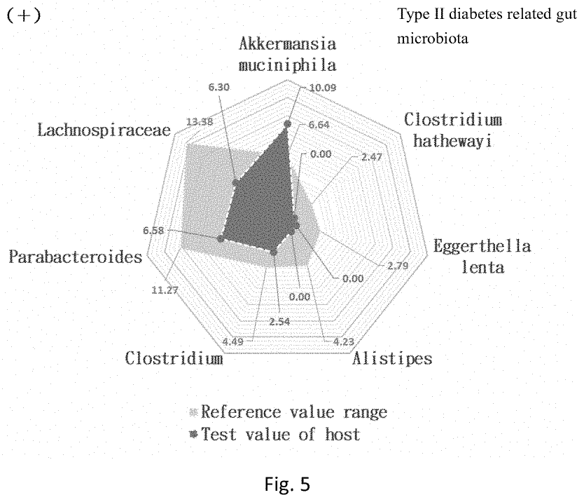

FIG. 6 is a negatively correlated gut microbiota radar chart of type II diabetes related gut microbiota in a stool sample described in example 1;

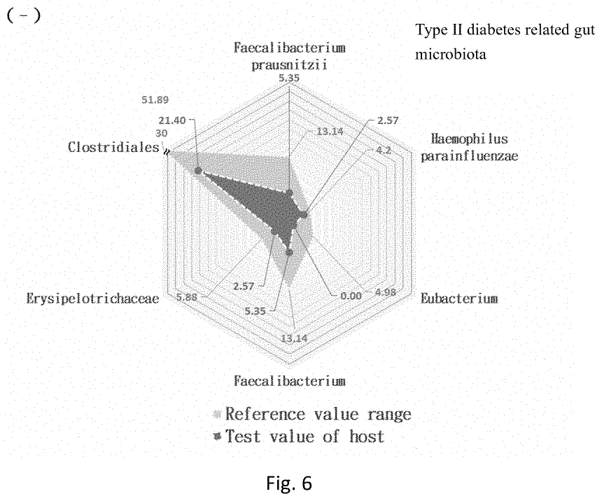

FIG. 7 is a positively correlated gut microbiota radar chart of type I diabetes related gut microbiota in a stool sample described in example 1;

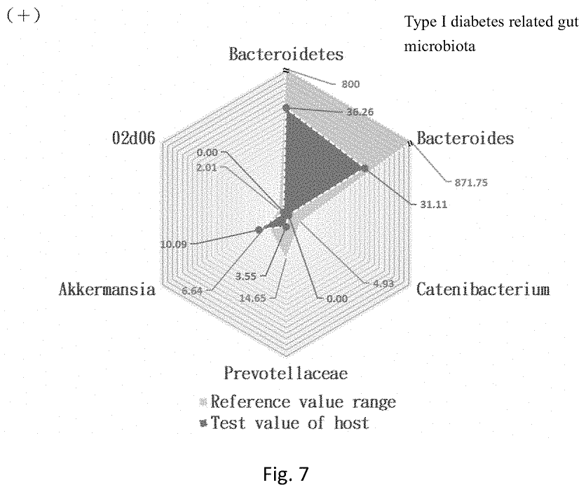

FIG. 8 is a negatively correlated gut microbiota radar chart of type I diabetes related gut microbiota in a stool sample described in example 1;

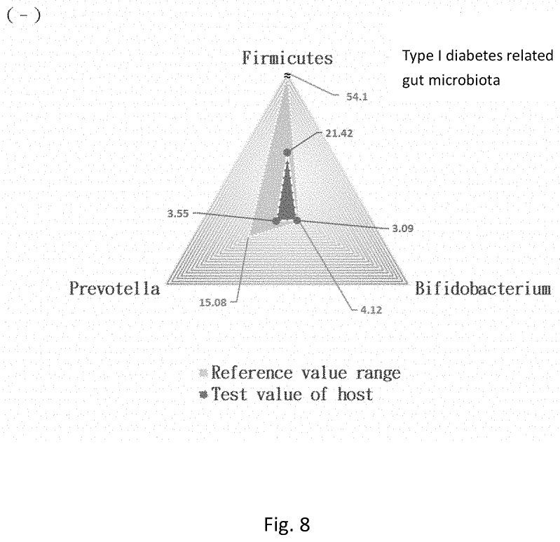

FIG. 9 is a positively correlated gut microbiota radar chart of Crohn's disease related gut microbiota in a stool sample described in example 1;

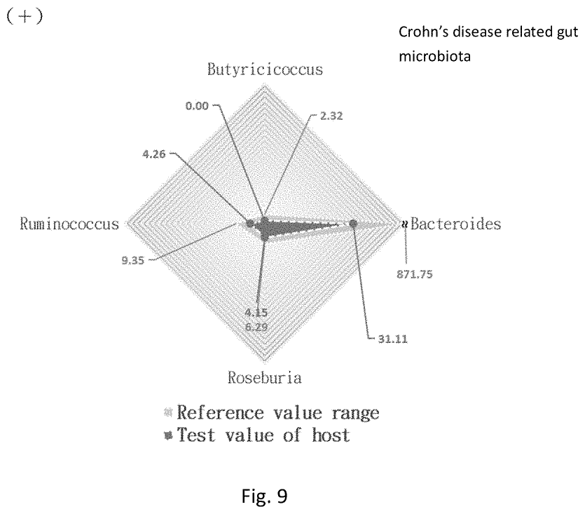

FIG. 10 is a negatively correlated gut microbiota radar chart of Crohn's disease related gut microbiota in a stool sample described in example 1;

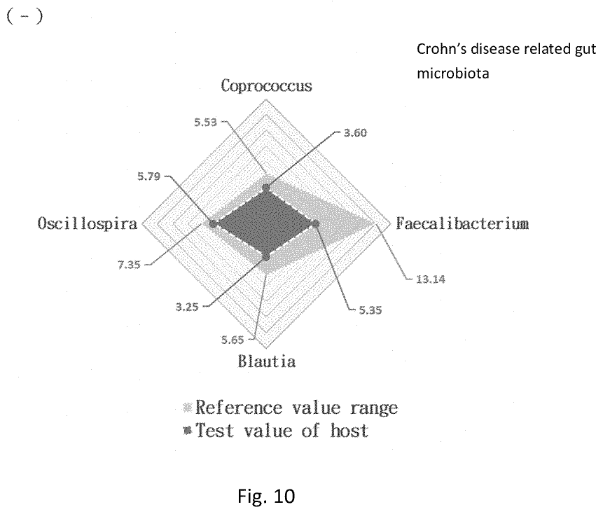

FIG. 11 is a radar chart of depressive disorder related gut microbiota in a stool sample described in example 1;

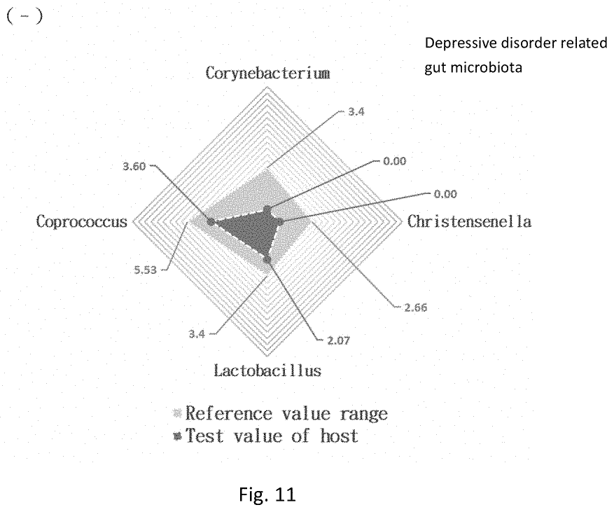

FIG. 12 is a plot illustrating diversity index of the gut microbiota in a stool sample described in example 1; and

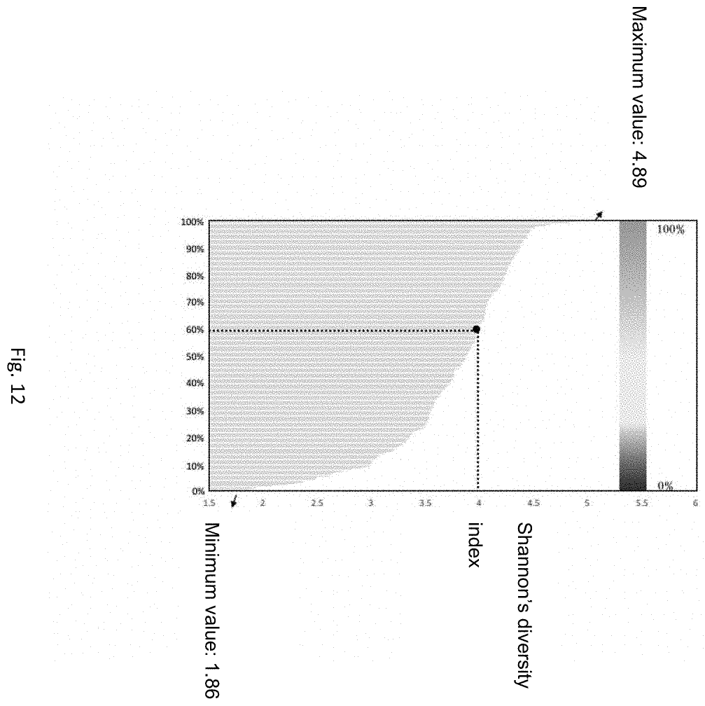

FIG. 13 is a plot illustrating enterotypes of the gut microbiota in a stool sample described in example 1.

### BRIEF DESCRIPTION OF THE PREFERRED EMBODIMENTS

What probed into the invention is a method for separating nanoparticles with a controlled number of active groups. Detailed descriptions of the production, structure and elements will be provided in the following in order to make the invention thoroughly understood. Obviously, the application of the invention is not confined to specific details familiar to those who are skilled in the art. On the other hand, the common elements and procedures that are known to everyone are not described in details to avoid unnecessary limits of the invention. Some preferred embodiments of the present invention will now be described in greater detail in the following. However, it should be recognized that the present invention can be practiced in a wide range of other embodiments besides those explicitly described, that is, this invention can also be applied extensively to other embodiments, and the scope of the present invention is expressly not limited except as specified in the accompanying claims.

In a first embodiment, the present invention discloses a method of building gut microbiota database. In detail, the method comprises following steps

Step 1: Provide a stool sample having an amount more than 500 mg.

Step 2: Extract the DNA containing gut microbiota from the stool sample by a kit (QIAGEN; QIAmp Fast DNA Stool Mini). After isolation, amount of the DNA is about 10˜50 ug and concentration of the DNA is about 50˜150 ng/ul. Perform PCR amplifying and adjust the concentration of the DNA to 50 ng/ul. Use a pair of primers consisting of a forward primer and a reverse primer that complement to V3˜V4 regions of 16 S rRNA gene among the bacterial DNA sequence, and amplifying the V3˜V4 sequence of 16 S rRNA gene of the gut microbiota to a length of about 550 bps. Purify the amplifying product by a kit (Geneaid; GeneHlow Gel/PCR). The sequence of the amplifying product has Illumina overhang adapters at the end of the sequence. Hence, use another kit (Nextera XT Index) containing a forward/reverse primers (P5, P7) to further amplify the sequence by PCR and purify the sequence by magnetic beads (AMPure XP) to obtain a library with a length of about 630 bp.

Step 3: Measure the size and concentration of the library by the analyzer and fluorometer (Agilent bioanalyzer 2100), adjust the concentration of the library, and add the library onto a sequencing chip with complementary adaptors on surface of the sequencing chip. Perform a bridge amplification by gene sequencing platform to amplify fluorescent detecting signals; and remove fluorescent markers and detect the fluorescent signals repeatedly to obtain gene sequencing information of the gut microbiota. The gene sequencing information of the gut microbiota has a pair-end sequencing length of about 2*300 bps.

Step 4: Input gene information obtained from step 2 to step 3 to a computer system install a metagenomics analyzing software (QIIME), a known gut microbiota gene database (Greengenes Database), and a disease related gut microbiota database. Execute the metagenomics analyzing software to generate group information of the gut microbiota by alignment and blasting algorithm, wherein the alignment and blasting algorithm calculate sequence similarity of each gene sequencing information and group gut microbiota data into operational taxonomic units when the sequence similarity of each gene sequencing information of the gut microbiota is more than 97%. Correspond the operational taxonomic units of the gut microbiota to taxonomic information stored in the known gut microbiota gene database and output the species of the gut microbiota and abundance of the species of the gut microbiota. The known gut microbiota gene database has Kingdom, Phylum, Class, Order, Family, Genus and Species bioinformation of the known gut microbiota.

Representatively, the gene sequencing is next generation gene sequencing.

Step 5: Cross-compare the species of the gut microbiota with the disease related gut microbiota database, and output classifying information of related gut microbiota by the computer system. The disease related gut microbiota comprises cancer related gut microbiota, cardiovascular disorders related gut microbiota, metabolism related gut microbiota, autoimmune related gut microbiota, gastrointestinal disorders related gut microbiota or mental/psychiatric related gut microbiota; and further classifying the related gut microbiota into positively correlated or negatively correlated gut microbiota.

Step 6: Calculate and output test value R(x) of the species of the gut microbiota that correlated to each diseases by the computer system.

The test value R(x) is obtained from following equation

$$R ( x ) = \frac{10}{- {log}_{10} x}$$

x is the abundance of the species of the gut microbiota.

Step 7: Calculate and output reference value range of the species of the gut microbiota that correlated to each diseases by the computer system, wherein the reference value range of the species of the gut microbiota is calculated by following equation.

$$Reference value range of the species of the gut microbiota = \frac{10}{- {log}_{10} ( x - 2 std )} ∼ \frac{10}{- {log}_{10} ( x + 2 std )}$$

x is the abundance of the species of the gut microbiota.

std is standard deviation of the abundance.

Step 8: Output a seties of radar charts by the computer system, wherein the seties of radar charts include following information: the classifying information of related gut microbiota, the test value R(x) of the species of the gut microbiota and the reference value range of the species of the gut microbiota that correlated to each disease by the computer system.

Step 9: Construct coordinates (x₀, y₀), (x₁, y₁), . . . , (xₙ₋₁, yₙ₋₁) and the corresponding polygon in the radar chart according to the aforementioned classifying information of related gut microbiota, test value R(x) of the species of the gut microbiota (Step 6) and reference value range of the species of the gut microbiota that correlated to each disease (Step 7) by the computer system. The area of the corresponding polygon (A) is calculated by following equation.

$$A = \frac{1}{2} ( \vert \begin{matrix}{x}_{0} & {x}_{1} \\ {y}_{0} & {y}_{1}\end{matrix} \vert + \vert \begin{matrix}{x}_{1} & {x}_{2} \\ {y}_{1} & {y}_{2}\end{matrix} \vert + ⋯ + \vert \begin{matrix}{x}_{n - 2} & {x}_{n - 1} \\ {y}_{n - 2} & {y}_{n - 1}\end{matrix} \vert + \vert \begin{matrix}{x}_{n - 1} & {x}_{0} \\ {y}_{n - 1} & {y}_{0}\end{matrix} \vert )$$

Step 10: Calculate and output a total protecting value (V) of the correlated gut microbiota by the computer system, wherein the total protecting value (V) of the correlated gut microbiota is an index for evaluating host health and calculated by following equation.

(V)=P[1−(Aₚ/Aₚᵣ)]+N[(Aₚ/Aₚᵣ)−1]

P is weights of the positively correlated gut microbiota in the related gut microbiota, 0≤P≤1; N is weights of the positively correlated gut microbiota in all the related gut microbiota, 0≤N≤1; Aₚ is area of the polygon constructed by the test value of the positively correlated gut microbiota; Aₚᵣ is area of the polygon constructed by the reference range of the positively correlated gut microbiota; Aₙ is area of the polygon constructed by the test value of the negatively correlated gut microbiota; Aₙᵣ is area of the polygon constructed by the reference range of the negatively correlated gut microbiota.

Step 11: Build the gut microbiota database according outputting results from step 5 to step 10 by the computer system. The gut microbiota database contains the classifying information of related gut microbiota, the test value R(x) of the species of gut microbiota in the related gut microbiota, the reference value range of the species of gut microbiota in the related gut microbiota, the total protecting value (V) of the related gut microbiota, evaluating index of diversity of the gut microbiota and enterotypes of the gut microbiota.

In one example of the first embodiment, if the total protecting value (V) of the related gut microbiota is more than or equal to zero, the computer system will output a result indicating normal host heath; and if wherein the total protecting value (V) of the related gut microbiota is less than zero, the computer system will output a result indicating abnormal host heath.

The total protecting value (V) of the related gut microbiota is related to the classifying information of related gut microbiota, the test value R(x) of the species of gut microbiota in the related gut microbiota, and the reference value range of the species of gut microbiota in the related gut microbiota.

The calculating equation of the total protecting value (V) of the related gut microbiota is as follows.

(V)=P[1−(Aₚ/Aₚᵣ)]+N[(Aₙ/Aₙᵣ)−1]

P is weights of the positively correlated gut microbiota in the related gut microbiota, and has a range of 0≤P≤1. For type II diabetes related gut microbiota, the P is 0.538. On the other hand, N is weights of the negatively correlated gut microbiota in all the related gut microbiota, and has a range of 0≤N≤1. For type II diabetes related gut microbiota, the N is 0.462.

The aforementioned positively correlated gut microbiota in the related gut microbiota is good gut microbiota for host. Oppositely, the negatively correlated gut microbiota in all the related gut microbiota is harmful gut microbiota for host.

In one example of the first embodiment, the cancer related gut microbiota comprises colorectal cancer related gut microbiota. The colorectal cancer related gut microbiota includes Fusobacterium, Helicobacter pylori and Bacteroides fragilis.

In one example of the first embodiment, the cardiovascular disorders related gut microbiota comprises atherosclerosis related gut microbiota. The atherosclerosis related gut microbiota includes positively correlated gut microbiota that comprises Oscillospira, Lachnospiraceae and Ruminococcus and negatively correlated gut microbiota that comprises Coriobacteriaceae, Erysipelotrichaceae and Allobaculum.

In one example of the first embodiment, the metabolism related gut microbiota comprises type II diabetes related gut microbiota. The type II diabetes related gut microbiota includes positively correlated gut microbiota that comprises Akkermansia muciniphila, Clostridium hathewayi, Eggerthella lento, Alistipes, Clostridium, Parabacteroides and Lachnospiraceae, and negatively correlated gut microbiota that comprises Faecalibacterium prausnitzii, Haemophilus parainfluenzae, Eubacterium, Faecalibacterium, Erysipelotrichaceae and Clostridiales.

In one example of the first embodiment, the autoimmune related gut microbiota comprises type I diabetes related gut microbiota. The type I diabetes related gut microbiota includes positively correlated gut microbiota that comprises Bacteroidetes, Bacteroides, Catenibacterium, Prevotellaceae, Akkermansia and code 02d06 belong to Greengenes Database, and negatively correlated gut microbiota that comprises Firmicutes, Bifidobacterium and Prevotella.

In one example of the first embodiment, the gastrointestinal disorders related gut microbiota comprises Crohn's disease related gut microbiota. The Crohn's disease related gut microbiota includes positively correlated gut microbiota that comprises Butyricicoccus, Bacteroides, Roseburia and Ruminococcus, and negatively correlated gut microbiota that comprises Coprococcus, Faecalibacterium, Blautia and Oscillospira.

In one example of the first embodiment, the mental/psychiatric related gut microbiota comprises depressive disorder related gut microbiota. The depressive disorder related gut microbiota includes Corynebacterium, Christensenella, Lactobacillus and Coprococcus.

In a second embodiment, the invention discloses a detecting system of gut microbiota. The detecting system of gut microbiota comprises, a sampler that collect stool sample, a gene information processing system, and a computer system. The gene information processing system comprises a gene library measuring unit and a gene sequencing unit. Typically, the computer system receive gene sequencing information output from the gene information processing system and execute the method described from paragraph [symbol] 0050 [symbol] to [symbol] 0069 [symbol] for building a gut microbiota database. The gut microbiota database comprises classifying information of related gut microbiota, test value R(x) of species of gut microbiota, reference value range of species of gut microbiota, analytical plots, total protecting value (V) of the related gut microbiota, evaluating index of diversity of the gut microbiota and enterotypes of the gut microbiota.

In one example of the second embodiment, the gene sequencing information is obtained by performing following steps: (1) extract bacterial DNA containing gut microbiota from a stool sample; (2) Perform PCR on the bacterial DNA containing gut microbiota from the stool sample to amplify the bacterial DNA; (3) For the PCR above, use a pair of primers consisting of a forward primer and a reverse primer that complement to V3-V4 regions of 16 S rRNA gene among the bacterial DNA sequence, and amplifying the V3-V4 sequence of 16 S rRNA gene of the gut microbiota to a length of about 550 bps; (4) Purify the V3-V4 sequence of 16 S rRNA gene of the gut microbiota and repeating step (2) and (3) to obtain a library with a length of about 630 bp; (5) Measure the size and concentration of the library by the analyzer and fluorometer, adjusting the concentration of the library, and adding the library onto a sequencing chip with complementary adaptors on surface of the sequencing chip; (6) Perform a bridge amplification by gene sequencing platform to amplify fluorescent detecting signals; and (7) Remove fluorescent markers and detect the fluorescent signals repeatedly to obtain gene sequencing information of the gut microbiota, wherein the gene sequencing information of the gut microbiota has a pair-end sequencing length of about 2*300 bp.

The detecting system of gut microbiota is applied to evaluate physiological conditions of a subject in vitro. The physiological conditions comprises physiological conditions of digestive system, physiological conditions of metabolic system, physiological conditions of immune system, physiological conditions of digestive tract cells, physiological conditions of central nervous system and/or physiological conditions of cardiovascular system.

Accordingly, the present invention provide the gut microbiota database with quantifiable indexes for evaluating host heath in vitro, and is able to apply in preventative and precision medicine.

In order to clearly interpret the method of building gut microbiota database with quantifiable indexes for evaluating host heath in vitro, the computer outputting results regarding to the gut microbiota are described in Example 1.

### Example 1

Collect a stool sample from a host first. Then, build gut microbiota database of the host according to the invented method of building gut microbiota database as described aforementioned.

TABLE 1 is a part of the gut microbiota database of the host and includes following information: classifying information of related gut microbiota, the test value R(x) of the species of gut microbiota in the related gut microbiota, and the reference value range of the species of gut microbiota in the related gut microbiota. Mark (+) represents positively correlated gut microbiota, and mark (−) represents negatively correlated gut microbiota.

TABLE 1

| | | |
|---|---|---|
| | | Reference |
| Gut microbiota | Test value | value range |

I-colorectal cancer related gut microbiota

| | | |
|---|---|---|
| Fusobacterium (+) | 0 | 0-4.71 |
| Helicobacter pylori (+) | 0 | 0-2.31 |
| Bacteroides fragilis (+) | 3.65 | 0-6.36 |

II-atherosclerosis related gut microbiota

| | | |
|---|---|---|
| Oscillospira (+) | 5.79 | 0-7.35 |
| Lachnospiraceae (+) | 6.30 | 0-13.38 |
| Ruminococcus (+) | 4.26 | 0-9.35 |
| Coriobacteriaceae (−) | 2.07 | 0-4.32 |
| Erysipelotrichaceae (−) | 2.57 | 0-5.88 |
| Allobaculum (−) | 0 | 0-2.25 |

III-type II diabetes related gut microbiota

| | | |
|---|---|---|
| Akkermansia_muciniphila (+) | 10.09 | 0-6.64 |
| Clostridium hathewayi (+) | 0 | 0-2.47 |
| Eggerthella lenta (+) | 0 | 0-2.79 |
| Alistipes (+) | 0 | 0-4.23 |
| Clostridium (+) | 2.54 | 0-4.49 |
| Parabacteroides (+) | 6.58 | 0-11.27 |
| Lachnospiraceae (+) | 6.30 | 0-13.38 |
| Faecalibacterium prausnitzii (−) | 5.35 | 0-13.14 |
| Haemophilus parainfluenzae (−) | 2.57 | 0-4.2 |
| Eubacterium (−) | 0 | 0-4.98 |
| Faecalibacterium (−) | 5.35 | 0-13.14 |
| Erysipelotrichaceae (−) | 2.57 | 0-5.88 |
| Clostridiales (−) | 21.40 | 0-51.89 |
| Akkermansia_muciniphila (−) | 10.09 | 0-6.64 |

IV-type I diabetes related gut microbiota

| | | |
|---|---|---|
| Bacteroidetes (+) | 36.26 | 18-800 |
| Bacteroides (+) | 31.11 | 8.91-871.75 |
| Catenibacterium (+) | 0 | 0-4.93 |
| Prevotellaceae (+) | 3.55 | 0-14.65 |
| Akkermansia (+) | 10.09 | 0-6.64 |
| 02d06 (+) | 0 | 0-2.01 |
| Firmicutes (−) | 21.42 | 0-54.1 |
| Bifidobacterium (−) | 3.09 | 0-4.12 |
| Prevotella (−) | 3.55 | 0-15.08 |

V-Crohn's disease related gut microbiota

| | | |
|---|---|---|
| Butyricicoccus (+) | 0 | 0-2.32 |
| Bacteroides (+) | 31.11 | 0-871.75 |
| Roseburia (+) | 4.15 | 0-6.29 |
| Ruminococcus (+) | 4.26 | 0-9.35 |
| Coprococcus (−) | 3.60 | 0-5.53 |
| Faecalibacterium (−) | 5.35 | 0-13.14 |
| Blautia (−) | 3.25 | 0-5.65 |
| Oscillospira (−) | 5.79 | 0-7.35 |

VI-depressive disorder related gut microbiota

| | | |
|---|---|---|
| Corynebacterium (−) | 0 | 0-3.4 |
| Christensenella (−) | 0 | 0-2.66 |
| Lactobacillus (−) | 2.07 | 0-3.4 |
| Coprococcus (−) | 3.6 | 0-5.53 |

TABLE 2 is another part of the gut microbiota database of the host and includes following information: weights of the positively or negatively correlated gut microbiota in the related gut microbiota (P and N), area of the polygons (Ap, An, Apr and Anr) and total protecting value (V) of the related gut microbiota.

TABLE 2

| | | | |
|---|---|---|---|
| Weights | Ap or An | Apr or Anr | (V) |

I-colorectal cancer related gut microbiota

| | | | | | | |
|---|---|---|---|---|---|---|
| 1 | (+) | 0.032 | (+) | 24.026 | (+) | 0.999 |

II-atherosclerosis related gut microbiota

| | | | | | | |
|---|---|---|---|---|---|---|
| 0.5 | (+) | 38.089 | (+) | 126.486 | (+) | −0.095 |
| 0.5 | (−) | 2.322 | (−) | 20.939 | (−) | |

III-type II diabetes related gut microbiota

| | | | | | | |
|---|---|---|---|---|---|---|
| 0.538 | (+) | 47.598 | (+) | 134.593 | (+) | −0.036 |
| 0.462 | (−) | 85.364 | (−) | 506.519 | (−) | |

IV-type I diabetes related gut microbiota

| | | | | | | |
|---|---|---|---|---|---|---|
| 0.667 | (+) | 504.375 | (+) | 1311.77 | (+) | 0.127 |
| 0.333 | (−) | 66.348 | (−) | 442.636 | (−) | |

V-Crohn's disease related gut microbiota

| | | | | | | |
|---|---|---|---|---|---|---|
| 0.5 | (+) | 73.61 | (+) | 255.489 | (+) | 0.022 |
| 0.5 | (−) | 38.141 | (−) | 114.513 | (−) | |

VI-depressive disorder related gut microbiota

| | | | | | | |
|---|---|---|---|---|---|---|
| 1 | (−) | 3.746 | (−) | 27.853 | (−) | −0.866 |

TABLE 3 is still another part of the gut microbiota database of the host and includes total protecting value (V) of the related gut microbiota, the host health condition in the related gut microbiota and marks. When the total protecting value (V) of the related gut microbiota is more than or equal to zero, the computer system output a result that indicates the host health in the related gut microbiota is normal and marks a green light. When the total protecting value (V) of the related gut microbiota is less than zero, the computer system outputs a result that indicates the host health in the related gut microbiota is abnormal. When the total protecting value (V) of the related gut microbiota is between 0 and −0.5, the computer system outputs a yellow light in the related gut microbiota. When the total protecting value (V) of the related gut microbiota is less than −0.5, the computer system outputs a red light in the related gut microbiota.

TABLE 3

| | | |
|---|---|---|
| (V) | Host heath | Mark |

I-colorectal cancer related gut microbiota

| | | |
|---|---|---|
| 0.999 | Normal | Green light |

II-atherosclerosis related gut microbiota

| | | |
|---|---|---|
| −0.095 | Abnormal | Yellow light |

III-type II diabetes related gut microbiota

| | | |
|---|---|---|
| −0.036 | Abnormal | Yellow light |

IV-type I diabetes related gut microbiota

| | | |
|---|---|---|
| 0.127 | normal | Green light |

V-Crohn's disease related gut microbiota

| | | |
|---|---|---|
| 0.022 | normal | Green light |

VI-depressive disorder related gut microbiota

| | | |
|---|---|---|
| −0.866 | Abnormal | Red light |

The index of diversity of the gut microbiota described in the example 1 is 3.98 that is more than 3.375, so the computer system output the index of diversity of the gut microbiota described in the example 1 to a high level.

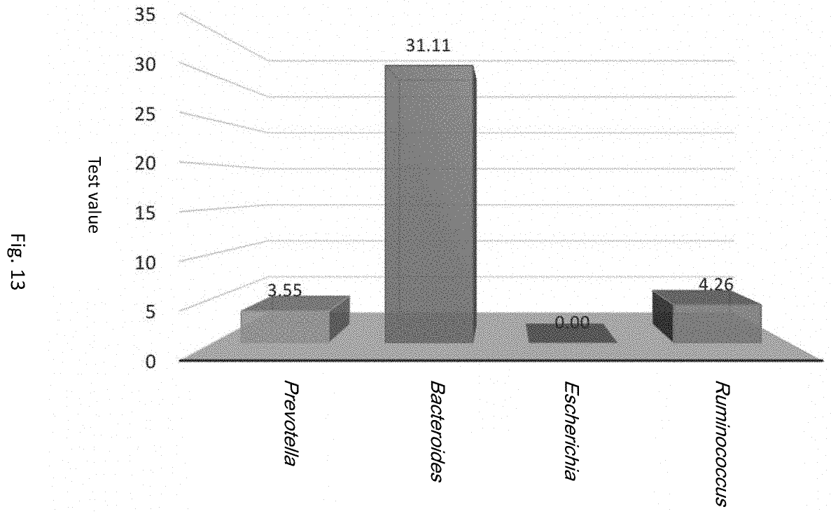

The enterotypes of the gut microbiota in the stool described in the example 1 is determined by whether the following gut microbiota has maximum test value R(x). The enterotypes of the gut microbiota in the stool described in the example 1 comprises Prevotella, Bacteroides, Escherichia, Ruminococcus as shown in FIG. 13. The test value R(x) of Bacteroides is 31.11 and is maximum when compare to others, so the enterotypes of the gut microbiota in the stool described in the example 1 is Bacteroides.

Accordingly, the present invention disclose a method of building gut microbiota database and detection system of gut microbiota. In particular, the method of building gut microbiota database is to provide the gut microbiota database with quantifiable indexes for evaluating host heath in vitro. The method apply gene sequencing method, especially next generation gene sequencing, and metagenomics analyzing technology to obtain gene information from the stool sample. Build the gut microbiota database by the aforementioned method and output the information by the computer system. The information comprises the classifying information of related gut microbiota, the test value R(x) of the species of gut microbiota in the related gut microbiota, the reference value range of the species of gut microbiota in the related gut microbiota, the seties of radar charts, the total protecting value (V) of the related gut microbiota, evaluating index of diversity of the gut microbiota and enterotypes of the gut microbiota.

Obviously many modifications and variations are possible in light of the above teachings. It is therefore to be understood that within the scope of the appended claims the present invention can be practiced otherwise than as specifically described herein. Although specific embodiments have been illustrated and described herein, it is obvious to those skilled in the art that many modifications of the present invention may be made without departing from what is intended to be limited solely by the appended claims.

What is claimed is:

1. A method of building gut microbiota database, comprising,
(1) Inputting gene sequencing information of gut microbiota to a computer system comprising a metagenomics analyzing software, a known gut microbiota gene database, and a disease related gut microbiota database;
(2) Classifying species of the gut microbiota with the computer system by following steps:
(I) Executing the metagenomics analyzing software to generate group information of the gut microbiota by alignment and blasting algorithm, wherein the alignment and blasting algorithm calculate sequence similarity of each gene sequencing information and group gut microbiota data into operational taxonomic units when the sequence similarity of each gene sequencing information of the gut microbiota is more than 97%;
(II) Corresponding the operational taxonomic units of the gut microbiota to taxonomic information stored in the known gut microbiota gene database; and
(III) outputting the species of the gut microbiota and abundance of the species of the gut microbiota;
(3) Cross-comparing the species of the gut microbiota with the disease related gut microbiota database, and outputting classifying information of related gut microbiota by the computer system, wherein the disease related gut microbiota comprises cancer related gut microbiota, cardiovascular disorders related gut microbiota, metabolism related gut microbiota, autoimmune related gut microbiota, gastrointestinal disorders related gut microbiota or mental/psychiatric related gut microbiota; and further classifying the related gut microbiota into positively correlated or negatively correlated gut microbiota;
(4) Outputting test value R(x) of the species of the gut microbiota that correlated to each diseases by the computer system, where
$$R ( x ) = \frac{10}{- {log}_{10} x}$$
and x is the abundance of the species of the gut microbiota;
(5) Outputting reference value range of the species of the gut microbiota that correlated to each diseases by the computer system, wherein the reference value range of the species of the gut microbiota is calculated by following equation:
$$Reference value range of the species of the gut microbiota = \frac{10}{- {log}_{10} ( x - 2 std )} ∼ \frac{10}{- {log}_{10} ( x + 2 std )}$$
where x is the abundance of the species of the gut microbiota and std is standard deviation of the abundance;
(6) Outputting a seties of radar charts by the computer system, wherein the seties of radar charts include following information: the classifying information of related gut microbiota, the test value R(x) of the species of the gut microbiota and the reference value range of the species of the gut microbiota that correlated to each disease by the computer system;
(7) Outputting a total protecting value (V) of the correlated gut microbiota by the computer system, wherein the total protecting value (V) of the related gut microbiota is an index for evaluating host health and calculated by following equation
(V)=P[1−(Aₚ/Aₚᵣ)]+N[(Aₙ/Aₙᵣ)−1]
where P is weights of the positively correlated gut microbiota in the related gut microbiota, 0≤P≤1;
N is weights of the positively correlated gut microbiota in all the related gut microbiota, 0≤N≤1;
Aₚ is area of the polygon constructed by the test value of the positively correlated gut microbiota;
Aₚᵣ is area of the polygon constructed by the reference range of the positively correlated gut microbiota;
Aₙ is area of the polygon constructed by the test value of the negatively correlated gut microbiota;
Aₙ, is area of the polygon constructed by the reference range of the negatively correlated gut microbiota; and
Building a gut microbiota database according outputting results from step (3) to step (7) by the computer system, wherein the gut microbiota database comprises the classifying information of related gut microbiota, the test value R(x) of the species of gut microbiota in the related gut microbiota, the reference value range of the species of gut microbiota in the related gut microbiota, the seties of radar charts, the total protecting value (V) of the related gut microbiota, evaluating index of diversity of the gut microbiota and enterotypes of the gut microbiota.

2. The method of claim 1, further comprises a step of extracting bacterial DNA containing gut microbiota from a stool sample and a sequencing process with bacterial gene amplification methods.

3. The method of claim 2, wherein the sequencing process with the bacterial gene amplification methods comprising following steps:
(1) Performing PCR on the bacterial DNA containing gut microbiota from the stool sample to amplify the bacterial DNA;
(2) For the PCR above, using a pair of primers consisting of a forward primer and a reverse primer that complement to V3-V4 regions of 16 S rRNA gene among the bacterial DNA sequence, and amplifying the V3-V4 sequence of 16 S rRNA gene of the gut microbiota to a length of about 550 bps;
(3) Purifying the V3-V4 sequence of 16 S rRNA gene of the gut microbiota and repeating step (2) and (3) to obtain a library with a length of about 630 bp;
(4) Measuring the size and concentration of the library by the analyzer and fluorometer, adjusting the concentration of the library, and adding the library onto a sequencing chip with complementary adaptors on surface of the sequencing chip;
(5) Performing a bridge amplification by gene sequencing platform to amplify fluorescent detecting signals; and
(6) Removing fluorescent markers and detecting the fluorescent signals repeatedly to obtain gene sequencing information of the gut microbiota, wherein the gene sequencing information of the gut microbiota has a pair-end sequencing length of about 2*300 bp

4. The method of claim 1, if wherein the total protecting value (V) of the related gut microbiota is more than or equal to zero, the computer system will output a result indicating normal host heath; and if wherein the total protecting value (V) of the related gut microbiota is less than zero, the computer system will output a result indicating abnormal host heath.

5. The method of claim 1, wherein the evaluating index of diversity of the gut microbiota is Shannon's diversity index which ranges from 1.86 to 4.89, if Shannon's diversity index of stool sample is more than or equal to 3.375, the stool sample is determined to a stool sample with high diversity of the gut microbiota; if Shannon's diversity index of stool sample is between 3.375 and 2.6175, the stool sample is determined to a stool sample with intermediate diversity of the gut microbiota; and if Shannon's diversity index of stool sample is less than or equal to 2.6175, the stool sample is determined to a stool sample with low diversity of the gut microbiota.

6. The method of claim 1, wherein the enterotypes of the gut microbiota comprises Prevotella, Bacteroides, Escherichia or Ruminococcus; and select the species of the gut microbiota having maximum test value of the species of gut microbiota to be the enterotypes of the gut microbiota in the stool sample.

7. The method of claim 1, wherein the gut microbiota database is save in a cloud storage and a gut microbiota detecting system.

8. A detecting system of gut microbiota, comprising, a sampler that collect stool sample, a gene information processing system, and a computer system; wherein the gene information processing system comprises a gene library measuring unit and a gene sequencing unit; and wherein the computer system receive gene sequencing information output from the gene information processing system and execute the method of claim 1 to build a gut microbiota database that comprises classifying information of related gut microbiota, test value R(x) of species of gut microbiota, reference value range of species of gut microbiota, analytical plots, total protecting value (V) of the related gut microbiota, evaluating index of diversity of the gut microbiota and enterotypes of the gut microbiota.

9. The detecting system of gut microbiota of claim 8, wherein the gene sequencing information is obtained by performing following steps:
(1) extracting bacterial DNA containing gut microbiota from a stool sample;
(2) Performing PCR on the bacterial DNA containing gut microbiota from the stool sample to amplify the bacterial DNA;
(3) For the PCR above, using a pair of primers consisting of a forward primer and a reverse primer that complement to V3-V4 regions of 16 S rRNA gene among the bacterial DNA sequence, and amplifying the V3-V4 sequence of 16 S rRNA gene of the gut microbiota to a length of about 550 bps;
(4) Purifying the V3-V4 sequence of 16 S rRNA gene of the gut microbiota and repeating step (2) and (3) to obtain a library with a length of about 630 bp;
(5) Measuring the size and concentration of the library by the analyzer and fluorometer, adjusting the concentration of the library, and adding the library onto a sequencing chip with complementary adaptors on surface of the sequencing chip;
(6) Performing a bridge amplification by gene sequencing platform to amplify fluorescent detecting signals; and
(7) Removing fluorescent markers and detecting the fluorescent signals repeatedly to obtain gene sequencing information of the gut microbiota, wherein the gene sequencing information of the gut microbiota has a pair-end sequencing length of about 2*300 bp.

10. The detecting system of gut microbiota of claim 8, being evaluated physiological conditions of a subject in vitro, wherein the physiological conditions comprises physiological conditions of digestive system, physiological conditions of metabolic system, physiological conditions of immune system, physiological conditions of digestive tract cells, physiological conditions of central nervous system and/or physiological conditions of cardiovascular system.
